(19) United States
(12) Patent Application Publication
(10) Pub. No.: US 2006/0068459 A1
(43) Pub. Date: Mar. 30, 2006

(54) Methods for detecting Xanthrochromia

(76) Inventor: Gregory D. Jay, Norfolk, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/237,347

(22) Filed: Sep. 28, 2005

Related U.S. Application Data
(60) Provisional application No. 60/614,127, filed on Sep. 28, 2004.

(51) Int. Cl. C12Q 1/04; A61M 31/00

(52) U.S. Cl. 435/34; 604/500

(57) ABSTRACT

The invention provides methods and compositions for making a diagnosis of Xanthrochromia in patient-derived samples of cerebrospinal fluid.

This application claims priority to U.S. provisional application 60/614,127 filed on Sep. 28, 2004, the entire contents of which are hereby incorporated by reference.

### BACKGROUND OF THE INVENTION

Headache accounts for approximately 2% of all emergency department (ED) visits. Although most headaches can be attributed to a benign cause, clinicians must be vigilant for potentially devastating subarachnoid hemorrhage (SAH). Each year approximately 30,000 patients in the United States suffer nontraumatic SAH. Alarmingly, an estimated 25% of SAH patients may be misdiagnosed upon initial presentation. Early diagnosis and treatment of SAH may avoid serious neurologic disability or death.

Xanthrochromia in cerebrospinal fluid (CSF) is indicative of SAH. Evaluation of a patient with severe headache in which SAH needs to be ruled out consists of urgent head CT scanning followed by CSF analysis in those patients who had a negative head CT study. A subset of patients with acute SAH do not evidence blood, but do reveal the presence of oxidized hemoglobin which is detected by laboratory personnel as Xanthrochromia.

### SUMMARY OF THE INVENTION

The invention is based on the discovery that increasing the diameter of a test tube for analyzing CSF enhances the ability of laboratory staff and emergency physicians who perform spinal taps to identify xanthrochromia and render a diagnosis of SAH. Accordingly, the invention provides a redesigned CSF collection tube which exceeds a critical diameter, permitting a large enough optical path length for the human eye to determine when CSF does not appear clear. The improved tube has an internal diameter that exceeds 1.3 cm. Larger diameter collection tubes or vials increase the ability of laboratory personnel to detect spectrophotometric xanthrochromia using standard visual inspection. Preferably, the diameter is 1.4 cm or greater. For example, the internal diameter of the tube in cm is 1.32, 1.35, 1.37, 1.4, 1.42, 1.45, 1.47, 1.5, 1.6, 1.7, 1.8, 1.9, 2.0, or more. Preferably, the internal diameter is 1.5 cm. The diameter is preferably less than 5.0 cm, e.g., the diameter is less than 3.0 cm. The thickness of the tube or container wall is preferably less than 7 mm, less than 5 mm, less than 2 mm, or less than 1 mm. The tube is characterized as having a longer optical path compared to the test tubes in a standard lumbar puncture kit. Typically, four tubes are filled in sequence following aspiration of cerebrospinal fluid and two of the tubes, e.g., tubes 1 and 4 of the sequence, are analyzed for Xanthrochromia. Standard spinal tap kits typically contain 4 to 5 tubes or vials. An improved kit includes a tube with a diameter of greater than 1.3 cm in positions designated for Xanthrochromia analysis in the sampling sequence. For example, an improved lumbar puncture kid contains Tube 1 for cell count or Xanthrochromia analysis, Tube 2 for stat gram stain and culture (C+S), Tube 3 for glucose or protein determination, Tube 4 for cell count or Xanthrochromia analysis (for comparison to Tube 1), and Tube 5 (optional) for virology, mycology, cytology, etc. in which the diameter of the cell those tubes designated for Xanthrochromia analysis have an internal diameter that is greater than 1.3 cm. Optionally, the CSF containers for Xanthrochromia analysis are configured such that the lower portion of the container is in the shape of an optical cuvette (1 cm path length) to allow both visual and spectrophotometric analysis of the CSF sample.

The test tube or specimen collection vessel has a tube body of unitary construction. The tube body is preferably plastic (e.g., polypropylene, polyurethane, or polystyrene); however, other compositions such as glass or quartz are also used. Any clear plastic or optically transparent polymeric composition with low distortion properties are used to form the body of the tube. Any composition that is clear and has a refractive index in the range of 1.0 -1.6 is used to form the CSF container. For example, the refractive index is about 1.5-1.6 (refractive index of glass), e.g. 1.46 (refractive index of quartz). Preferably, the refractive index of the container or tube body is less than that of bodily fluids such as CSF. The refractive index is in the range of 1.3-1.5 (refractive index of water or lightly buffered saline is about 1.33). The tube is useful for visual inspection and evaluation of CSF and other bodily fluids for changes in color or other parameters compared to a normal nonpathologic (e.g., clear) control fluid such as water or phosphate buffered saline (PBS).

Accordingly, the invention provides a lumbar puncture kit for use in detecting Xanthrochromia in cerebrospinal fluid. The kit contains a first and second container, each for use in analyzing spinal fluid and a third and fourth container, each configured to hold spinal fluid for analysis for Xanthrochromia. The third and fourth containers are configured to be transparent along optical path lengths through the third and fourth containers to allow visual inspection of the spinal fluid through each of the third and fourth containers, and the third and fourth containers are configured to hold the spinal fluid such portions of the path lengths occupied by the spinal fluid in each of the third and fourth containers measure greater than 1.3 cm. For example, the third and fourth containers are circular test tubes with inner diameters each greater than 1.3 cm. Preferably, the inner diameter is 1.5 cm. In another example, the third and/or fourth container includes substantially flat inner and outer walls defining the optical path. The first container of the kit is designated for use in microbiological analysis and the second container is designated for use in glucose and/or protein determination. The third and/or fourth container optionally contains a reference color disposed in close proximity to the optical path. Preferably, the reference color is white.

Also within the invention is a Xanthrochromia analysis container. The container includes the following features: a spectrographic inspection segment comprising first transparent walls defining a first path length inside the container approximately 1 cm long, the spectrographic inspection segment configured to be inserted into and retained by a spectrographic inspection station; and a visual inspection segment connected to the spectrographic segment and comprising second transparent walls defining a second path length inside the container greater than 1.3 cm long. The visual inspection segment is circular, with an inner diameter greater than about 1.3 cm. In preferred embodiments, the inner diameter is greater or equal to than 1.5 cm. In some embodiments, opposing wall portions of the visual inspection segment have substantially flat inner and outer surfaces. Preferably, the visual inspection segment comprises material with a refractive index less than a refractive index of spinal fluid. An alternative embodiment of the container has a visual inspection segment that includes a divider wall separating a region configured to contain the spinal fluid and a region configured to be devoid of the spinal fluid.

A method of detecting Xanthrochromia is carried out by visually inspecting a sample of patient-derived cerebrospinal fluid in a transparent container by looking through the container along a linear sight path that extends through greater than 1.3 cm of the fluid. The method may also include the steps of removing cerebrospinal fluid from a patient, placing the fluid in a transparent container, and then visually inspecting the fluid in the container by looking through the container along a linear sight path that extends through greater than 1.3 cm of the fluid. Preferably, the linear sight path extends through about 1.5 cm of the fluid. The method optionally includes a step of comparing a color of the fluid with a color reference that is laterally displaced relative to, and in close proximity to, the sight path.

Other embodiments and features of the invention will be apparent from the following description thereof, and from the claims.

### BRIEF DESCRIPTION OF THE DRAWINGS

FIG. 1 is a line graph showing a CSF oxyhemoglobin curve compared with CSF control.

FIG. 2 is a diagram of a Xanthrochromia analysis container with a visual segment and a spectrophotometric segment.

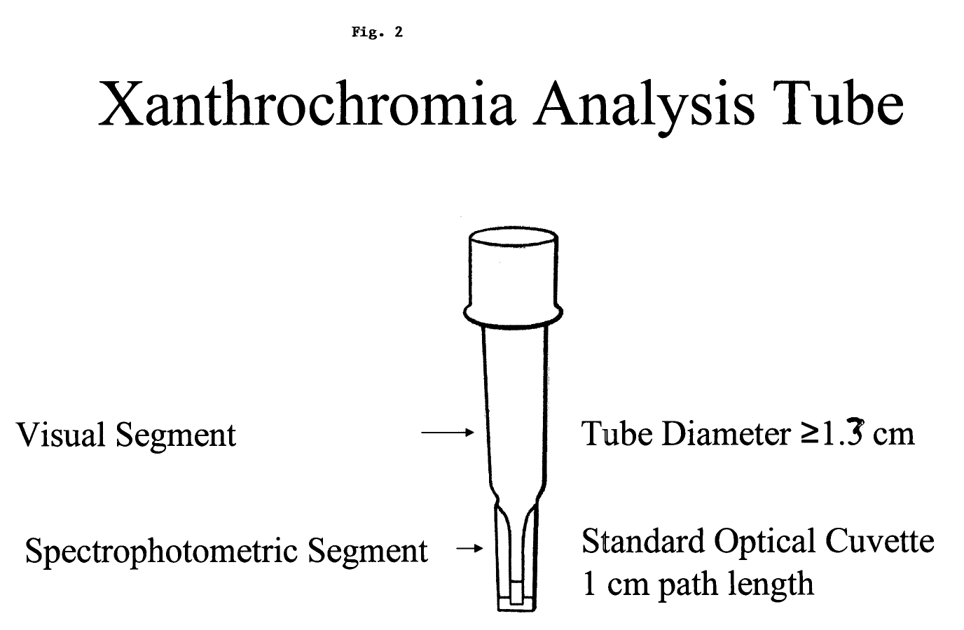

FIG. 3 is a diagram of a Xanthrochromia analysis container with an inner diameter of greater than 1.3 cm.

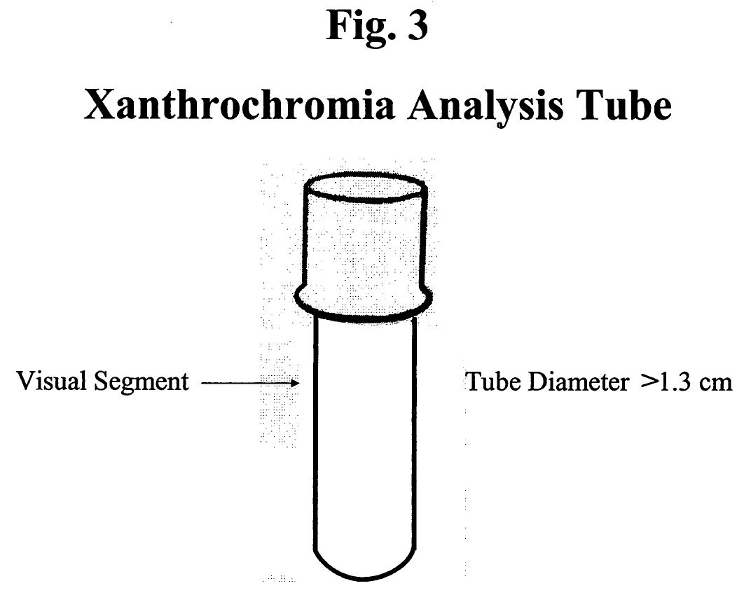

FIG. 4 is a perspective view of an alternative CSF analysis container for Xanthrochromia detection.

### DETAILED DESCRIPTION

When SAH is suspected, evaluation often begins with an emergent noncontrast computerized tomography (CT) scan of the brain. Ideally, a normal imaging study would exclude SAH, but this is not the case. It is recommended that a patient being evaluated for SAH should undergo lumbar puncture (LP) in the setting of a normal CT scan.

CSF analysis in patients with suspected SAH consists of two steps: (1) quantifying the number of red blood cells (RBCs) and (2) determining the presence or absence of xanthrochromia. Xanthrochromia is the yellowish discoloration of the CSF supernatant caused by the breakdown products of hemoglobin (oxyhemoglobin and bilirubin). The RBC count alone can be unreliable for several reasons. First, RBCs can be introduced into the CSF by traumatized adjacent soft tissue thereby falsely increasing the RBC count. Although it can be helpful to note the decrease in the number of RBCs in successive tubes, the possibility of a concomitant traumatic tap and SAH still exists. Second, there is no defined threshold for the number of RBCs in CSF to establish a diagnosis of SAH. Finally, while all SAH patients presenting early are expected to have RBCs in their spinal fluid, the percentage of patients with non-bloody CSF increases over time; after 12 hours from the onset of headache, some patients may have non-bloody CSF. For these reasons, spinal fluid RBC count is augmented by determining the presence or absence of xanthrochromia.

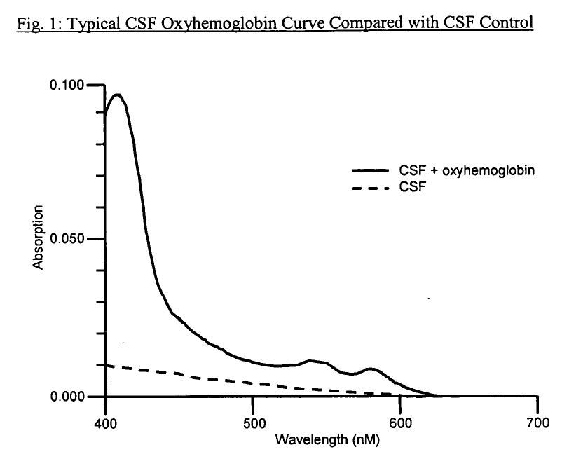

Spectrophotometry is the recommended technique for determining the presence or absence of xanthrochromia. Practice guidelines of the Association of Clinical Pathologists recommend scanning CSF supernatant using a double beam spectrophotometer at wavelengths between 350 nm and 650 nm. Xanthrochromia is defined as an absorbance of greater than 0.023 at 415 nm (oxyhemoglobin peak) and/or a bilirubin peak in the absorbance curve at 450-460 nm. A typical oxyhemaglobin absorbance curve compared to clear CSF is shown in FIG. 1. Using spectrophotometric analysis, Xanthrochromia is considered present if the absorption is greater than 0.023 at 415 nm.

Despite the assertion that spectrophotometry is the ideal method of detecting xanthrochromia, almost all hospital laboratories in the United States use visual inspection for the determination of xanthrochromia. A telephone survey of 805 hospital directors in the year 2000 found that only eight used spectrophotometry. In 2001, a mail survey of almost 2000 hospital clinical laboratory directors also identified only eight hospitals using spectrophotometry for xanthrochromia.

As 99% of laboratories in the U.S. use visual inspection for detection of xanthrochromia, the sensitivity of visual detection was evaluated. The hypothesis was that the human eye is not sensitive enough to detect spectrophotometricaly defined xanthrochromia. Studies were carried out to determine whether the sensitivity of visual detection could be improved by either using a reference tube of fluid just over the spectrophotometric threshold, or by increasing the diameter of the tube through which the CSF is viewed.

A Comparison of Visual and Spectrophotometric Xanthrochromia.

Most U.S. hospitals use visual inspection for the detection of CSF xanthrochromia. Visual inspection was compared with spectophotometric methods of detecting xanthrochromia, and the effect of tube diameter on the sensitivity of visual inspection was evaluated.

Blinded, experienced laboratory technicians visually examined unmarked samples to determine the presence or absence of xanthrochromia. Samples were prepared by lysing red blood cells in distilled water. Serial dilutions were placed in clear polystyrene tubes obtained from standard lumbar puncture trays. Laboratory technicians were asked to examine each sample for xanthrochromia using visual inspection. Next, they were then asked to interpret the same set of samples with the assistance of a threshold standard. Lastly, they were asked to interpret the same dilutions, but this time presented in a larger diameter tube. The absorbance of each sample was measured in a double beam spectrophotometer at wavelengths between 300 nm and 700 nm. Samples were said to demonstrate spectrophotometric xanthrochromia if they had an absorbance greater than 0.023 at 415 nm.

The following results were obtained from the study. Sixteen laboratory technicians were shown a total of 160 samples, of which 64 (40%) demonstrated spectrophotometric xanthrochromia. Visual inspection of the samples was 26.6% sensitive and 97.9% specific for spectrophotometric xanthrochromia. Using a reference standard did not improve performance (sensitivity 30%, specificity 98%, ppv 90%, npv 68%), but increasing the diameter of the collection tubes did improve sensitivity (sensitivity 55%, specificity 98%, ppv 95%, npv 76%).

The results indicated that visual inspection is not sensitive for the detection of spectrophotometric xanthrochromia. Increasing the diameter of the collection tubes did improve sensitivity.

The following methods were used to conduct the analysis.

Survey of Laboratory Practice

Laboratory technicians in a large tertiary care facility who routinely examine CSF samples for the presence of xanthrochromia consented to participate in this voluntary study. The study took place in the clinical laboratory in which the technicians worked. Prior to data collection, the technicians completed a questionnaire which asked about training backgrounds and professional experience. In three separate trials, the technicians were asked to determine the presence or absence of xanthrochromia in samples prepared with various dilutions of red blood cells lysed in deionized water.

Samples were prepared from blood drawn from a human subject, whose RBC count was determined to be 5.2×10⁹ RBC/ml. Three ml of this blood was vigorously mixed with three milliliters of deiodinated water to lyse the RBCs. A sample containing just deionized water and nine further dilutions were created by mixing the following proportions of serum to deiodinated water: none, 1 to 100,000, 1 to 80,000, 1 to 60,000, 1 to 50,000, 1 to 40,000, 1 to 30,000, 1 to 20,000, 1 to 10,000 and 1 to 5,000. This yielded samples which contained varying concentrations of RBC's from none to 1,000 RBCs/mm3 (Table 1).

A total of 0.5 cc of each dilution was microcentifuged for 10 minutes and then analyzed in a cuvette with a spectrophotometer (Spectronic Genesys 5, Spectronic Instruments, Rochester, N.Y.). Absorption units were recorded for peaks at 415 nm and 450 nm. Survey scans were performed in the range of 300 nm-700 nm for all samples and the resulting graphs were analyzed for stability testing. The samples were stored in polyurethane test tubes at room temperature and in darkness to prevent degradation of the RBC breakdown products. Samples were prepared and stored for two to four days before they were interpreted by the laboratory technicians. Every dilution was tested spectrophotometrically when it was made and just prior to technician interpretation. There was no change in the shape of the absorbance curve or the amplitude of any of the samples. Prior to conducting the study, a set of dilutions identical to the ones used in the study was created and serially tested on the spectrophotometer out to sixty days. No significant change in survey scans or maximal absorbance at 415 or 450 nm was observed.

For the first two trials, two milliliters of each dilution were placed into clear polystyrene tubes with an internal diameter of 1.3 cm obtained from the Allegience lumbar puncture trays #4301C (Allegience Healthcare Corporation, McGaw Park, Ill.). Each tube was assigned a random number known only to investigators. The ten tubes, containing the nine different dilutions of RBC's and a tube of pure water, were placed into a dark container. Three separate sets of ten tubes were made. For the third, the same set of dilutions was prepared in Fisherbrand (Fisher Scientific International Inc., Hampton, N.H.) clear polystyrene tubes with an internal diameter of 1.5.

In the first trial, technicians inspected tubes selected in random order from an opaque bag and were asked to use their usual method to determine if the sample was xanthrochromic. The investigator recorded each technician's technique. After each recording, the technician returned the sample to another opaque bag, prior to selecting the next tube. This approach was continued until all tubes were visually inspected.

The second trial was performed in identical fashion to the first, except the technicians were given a reference tube with a known absorbance of 0.027@415 nm and a piece of white paper to use as a background (Note: all technicians used a white background by choice in the first trial). The technicians were told this sample was positive for xanthrochromia by spectrophotometry, and they were asked to use the reference tube and white paper when analyzing the samples. They again randomly selected tubes from the bag of samples, and record their results.

In the third trial, the technicians read the samples prepared in the larger diameter tubes using their standard method, and recording their results in similar fashion.

The sensitivity and specificity of the laboratory technicians determination of xanthrochromia and the 95% confidence intervals were calculated.

Sixteen laboratory technicians were available during the study period, and all gave their consent to participate. All were credentialed to evaluate cerebrospinal fluid samples for the presence of xanthrochromia. The average length of clinical experience was 23 years with a range of 1.5 to 37.5 years. All sixteen technicians were women, and all were observed to use a white paper background in their determination of xanthrochromia. Seven of the 16 also used a tap water filled tube as part of their standard method. All indicated on a questionnaire that they had received training in detecting xanthrochromia.

Of the 64 viewings of samples that had spectrophotometric xanthrochromia in the first trial, xanthrochromia was only thought to be present in 17. Visual xanthrochromia was determined to be absent in 94 of 96 of the samples where spectrophotometric xanthrochromia was not present. (sens 27%, spec 98%, ppv 89%, npv 67%).

In the second trial, with the provision of a reference sample, 19 of 64 were correctly identified as xanthrochromic, and 94 of 96 were correctly identified as not demonstrating xanthrochromia. (sens 30%, spec 98%, ppv 90%, npv 68%).

In the third trial, using larger diameter tubes, of the 64 samples with spectrophotometric xanthrochromia, 35 were correctly identified as being xanthrochromic by visual detection, and of the 96 without spectrophotometric xanthrochromia, 94 were identified as negative. (sens 55%, spec 98%, ppv 95%, npv 76%). The results are summarized in Table 1.

Comparison of Visual Inspection for Xanthrochromia to the Spectrophotometric Detection of Xanthrochromia

Laboratory technicians from a tertiary care hospital visually examined unmarked samples for the presence or absence of xanthrochromia. Samples were prepared by vigorously mixing 3 mL of blood with 3 mL of deiodinated water to lyse the RBCs. Dilutions were then made ranging from 1 in 5,000 to 1 in 100,000. Samples of each dilution were placed into clear polystyrene tubes obtained from lumbar puncture trays. In a random sequence, laboratory technicians visually examined each sample for xanthrochromia. In addition, each sample was scanned using a double-beam spectrophotometer (Spectronic Genesis 5, Spectronic Instruments, Rochester, N.Y.) at wavelengths between 300 nm and 700 nm. Absorption was recorded for peaks at 415 nm in allergenic units, the resonant wavelength for oxyhemoglobin. Samples were considered to demonstrate xanthrochromia by spectrophotometry if their absorbance was greater than 0.023 allergenic units at 415 nm.

Sixteen laboratory technicians examined a total of 160 samples, of which 64 (40%) demonstrated spectrophotometric xanthrochromia. Visual inspection of the samples was 27% sensitive (95% confidence interval [CI] 16% to 39%) and 98% specific (95% CI 93% to 100%) for spectrophotometric xanthrochromia.

Visual inspection was found not to be sensitive for the detection of xanthrochromia, thereby underscoring the limitations of the standard visual technique in their interpretation of the cerebrospinal fluid analysis of potential subarachnoid hemorrhage patients.

Visual Inspection of CSF is Not Sensitive for the Determination of Xanthrochromia as Defined by Spectrophotometry.

The average limit of detection of the eye appears to be equivalent to a concentration of oxyhemoglobin in the CSF with an absorbance of approximately 0.220 at 415 nm. Therefore, for CSF to appear xanthrochromic by visual inspection, it must contain approximately 10 times more oxyhemoglobin than would be needed to result in spectrophotometric xanthrochromia (0.023 at 415 nm).

The use of a spectrophotometricaly positive standard did not improve the sensitivity of visual inspection. However, as evidenced by the technicians ability to detect xanthrochromia at an equivalent absorbance of 0.097 at 415 nm, a wider diameter tube improves the sensitivity of visual inspection.

No prior study has attempted to improve the visual diagnosis of xanthrochromia by modifying laboratory technique. The finding that visual inspection of CSF is not as sensitive as spectrophotometry is in agreement with an earlier study 32 patients with CT confirmed CNS space bleeding (SAH, subdural hematoma or intracerebral hematoma) who underwent CSF analysis. All 32 patients had xanthrochromia by spectrophotometry, but only half of the patients had visible xanthrochromia.

Clinical studies of SAH suggest that spectrophotometric xanthrochromia is more sensitive for SAH than visual inspection of CSF. In a study of 111 patients with a brain CT positive for SAH, who had a LP performed between 12 hours and 7 days after the onset of headache, all patients had spectrophotometric xanthrochromia. In another study of 61 patients with headache and angiographically confirmed cerebral aneurysms, more than half (54%) did not have visually detectable xanthrochromia. The patients without visual xanthrochromia included both patients with a positive brain CT (76%) and patients with the LP delayed>24 hours after the onset of headache (58%). Unfortunately, neither of these studies directly compared spectrophotometric and visual analysis.

Current practice dictates that only patients with a normal brain CT receive a LP in the work-up of possible SAH. SAH patients with a normal brain CT likely have smaller bleeds, and this may impact the ability to detect xanthrochromia. In a study of 79 patients with “worst” headache, no focal neurologic deficits, and a normal brain CT, 20 patients had xanthrochromia by spectrophotometry. Of these, only 2 (10%) also had xanthrochromia by visual inspection. Two patients had SAH. Both patients with SAH had spectrophotometric xanthrochromia, but, only one of these two SAH patients had visually detectable xanthrochromia.

Visual inspection is not sensitive for spectrophotometric xanthrochromia, and spectrophotometric xanthrochromia may be more sensitive for SAH than visual detection. Most hospitals do not analyze CSF by spectrophotometry. The study described herein indicates that the visual detection of xanthrochromia is improved by inspecting CSF in tubes with a larger diameter. The longer optical path length allows the visual detection of xanthrochromia that otherwise would only be observable with a spectrophotometer. Adoption of this technique facilitates detection of xanthrochromia.

Improving Visual Detection of Xanthrochromia Using a Larger Tube Diameter

Standard CSF collection tubes are polystyrene lumbar puncture sample containers with internal diameter approximately 1.3 cm. These standard tubes artifactually suppress the ability of the human eye to detect xanthrochromia leading to medical errors, e.g., erroneously dicharged patients following a workup for SAH in an emergency department.

Visual inspection for xanthrochromia does not replicate spectrophotometric determination of xanthrochromia. The use of a laboratory cut-off standard under ideal circumstances did not improve detection, but the use of a wider tube (i.e., greater than 1.3 cm in diamater) did improve the visual detection of xanthrochromia. A larger tube diameter did increase the sensitivity of visual detection of xanthrochromia, Because 99% of laboratories in the United States use visual inspection for detection of xanthrochromia, use of CSF tubes with a diameter of greater than 1.3 cm diameter is important to enable accurate naked eye detection of xanthrochromia. In an improved lumbar puncture kit, this larger tube replaces one of the 4 tubes in a standard CSF collection tube kit. Sensitivity is improved by using such larger diameter tubes.

CSF Collection Tube with Visual and Spectrophometric Analysis Segments

A cylindrical tube with diameter of 1.3 cm or greater is fitted with a standard plastic cuvette at the bottom. This top portion of the collection vessel is a cylindrical tube shape and the bottom portion of the vessel is a cylindrical or in square cuvette shape suitable for installation into a spectrophotometer. A collection vial configured in the manner allows the human visual inspection down the optical axis of this CSF collection tube as well as placing the tube into a spectrophotometer for spectrophotometric analysis, thereby eliminating the need for removing a CSF sample to a separate cuvette or tube for placement into a spectrophotometric device.

### OTHER EMBODIMENTS

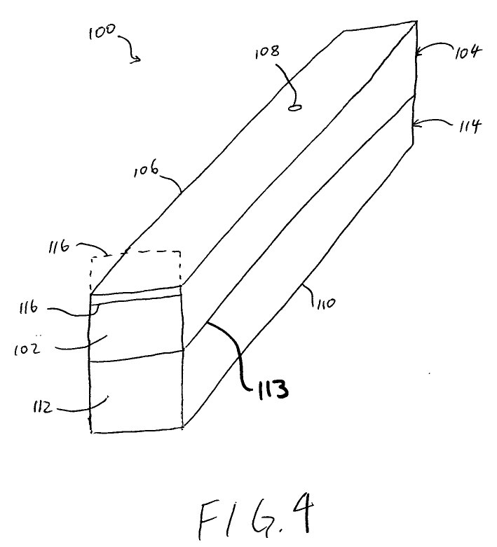

Various alternative containers or vessels may be used for screening for Xanthrochromia. For example, referring to FIG. 4, a container or vessel 100 is an elongated container with a rectangular cross-section. The container 100 has opposing end walls 102, 104 that are transparent (e.g., made of clear plastic, glass, quartz, etc.). The container 100 includes a top chamber 106 that is configured to hold spinal fluid. A hole 108 in a wall of the top chamber 106 provides access for filling the top chamber 106 with fluid. The hole 108 can be sealed to inhibit the fluid from escaping the chamber 106 while the fluid is observed. The container 100 includes a bottom chamber 112 separated from the top chamber 106 by a divider wall 113. The bottom chamber 110 includes transparent end walls 112, 114 and is preferably sealed and preferably is either devoid of contents (i.e., there is a vacuum inside) or contains air. The bottom chamber 110 can be used as a reference when viewing fluid in the top chamber 106. A color reference (e.g., white) may be applied to a portion of the wall 112, or may be provided elsewhere on the container 100, preferably adjacent to the end wall 102, e.g., above the end wall 102 as shown in dotted lines in FIG. 4. The container preferably has a length 116 of at least 1.3 cm, and preferably 1.5 cm, although longer lengths may be used. In use to detect the existence of Xanthrochromia, spinal fluid is placed in the chamber 102, and the container 100 is held with light behind the end wall 104 and the fluid viewed through the end walls 102 and 104. Any color of the fluid is ascertained, possibly with the help of the chamber 110 and/or the color reference. From this inspection, the user determines whether Xanthrochromia is present in the fluid.

TABLE 1
Interpretation of Fluid for the Presence of Xanthrochromia by Laboratory
Technicians.

| | | | Trial #1 (n = 16) | | Trial #2 (n = 16) | | | |
|---|---|---|---|---|---|---|---|---|
| | | | Routine | | With a | | Trial #3 (n = 16) | |
| Sample characteristics | | | interpretation | | ref. standard | | Larger tubes | |
| Serum Dil. | RBCs/mm3 | Abs. @ 415 nm | present | absent | present | absent | present | absent |
| no serum | 0 | 0.000 | 0 | 16 | 1 | 15 | 1 | 15 |
| 1 to 100,000 | 52 | 0.000 | 1 | 15 | 0 | 16 | 0 | 16 |
| 1 to 80,000 | 65 | 0.001 | 0 | 16 | 1 | 15 | 0 | 16 |
| 1 to 60,000 | 87 | 0.008 | 0 | 16 | 0 | 16 | 1 | 15 |
| 1 to 50,000 | 104 | 0.014 | 0 | 16 | 0 | 16 | 0 | 16 |
| 1 to 40,000 | 121 | 0.017 | 1 | 15 | 0 | 16 | 0 | 16 |
| 1 to 30,000 | 174 | *0.034 | 0 | 16 | 1 | 15 | 1 | 15 |
| 1 to 20,000 | 261 | *0.037 | 1 | 15 | 0 | 16 | 3 | 13 |
| 1 to 10,000 | 522 | *0.097 | 3 | 13 | 4 | 12 | 15 | 1 |
| 1 to 5,000 | 1,040 | *0.220 | 13 | 3 | 14 | 2 | 16 | 0 |

*denotes xanthrochromia by spectrophotometry.

1. A lumbar puncture kit for use in detecting Xanthrochromia in cerebrospinal fluid, the kit comprising:
first and second containers for use in analyzing spinal fluid; and
third and fourth containers configured to hold spinal fluid for analysis for Xanthrochromia;
wherein the third and fourth containers are configured to be transparent along optical path lengths through the third and fourth containers to allow visual inspection of the spinal fluid through each of the third and fourth containers, wherein the third and fourth containers are configured to hold the spinal fluid such portions of the path lengths occupied by the spinal fluid in each of the third and fourth containers is greater than 1.3 cm.

2. The kit of claim 1 wherein the third and fourth containers comprise circular test tubes with inner diameters each greater than 1.3 cm.

3. The kit of claim 1 wherein the third and fourth containers comprise substantially flat inner and outer walls defining the optical path.

4. The kit of claim 1 wherein the first container is designated for use in microbiological analysis and the second container is designated for use in glucose or protein determination.

5. The kit of claim 1 wherein the third container comprises a reference color disposed in close proximity to the optical path.

6. The kit of claim 5 wherein the reference color is white.

7. A Xanthrochromia analysis container comprising:
a spectrographic inspection segment comprising first transparent walls defining a first path length inside the container approximately 1 cm long, the spectrographic inspection segment configured to be inserted into and retained by a spectrographic inspection station; and
a visual inspection segment connected to the spectrographic segment and comprising second transparent walls defining a second path length inside the container greater than 1.3 cm long.

8. The container of claim 7 wherein the visual inspection segment is circular, with an inner diameter greater than about 1.3 cm.

9. The container of claim 8 wherein the inner diameter is 1.5 cm.

10. The container of claim 7 wherein opposing wall portions of the visual inspection segment have substantially flat inner and outer surfaces.

11. The container of claim 7 wherein the visual inspection segment comprises material with a refractive index less than a refractive index of spinal fluid.

12. The container of claim 7 wherein the visual inspection segment includes a divider wall separating a region configured to contain the spinal fluid and a region configured to be devoid of the spinal fluid.

13. A method of detecting Xanthrochromia, comprising visually inspecting a sample of patient-derived cerebrospinal fluid in a transparent container by looking through the container along a linear sight path that extends through greater than 1.3 cm of the fluid.

14. The method of claim 13 wherein the linear sight path extends through about 1.5 cm of the fluid.

15. The method of claim 13 further comprising comparing a color of the fluid with a color reference that is laterally displaced relative to, and in close proximity to, the sight path.

16. A method of detecting Xanthrochromia in cerebrospinal fluid, the method comprising:
removing cerebrospinal fluid from a patient;
placing the fluid in a transparent container;
visually inspecting the fluid in the container by looking through the container along a linear sight path that extends through greater than 1.3 cm of the fluid.

17. The method of claim 16 wherein the linear sight path extends through about 1.5 cm of the fluid.

18. The method of claim 16 further comprising comparing a color of the fluid with a color reference that is laterally displaced relative to, and in close proximity to, the sight path.
